(19) United States
(12) Patent Application Publication
(10) Pub. No.: US 2025/0249274 A1
(43) Pub. Date: Aug. 7, 2025

(54) PHOTOTHERAPY DEVICE CONTROLLER

(71) Applicant: LUMITEX, INC., Strongsville, OH (US)

(72) Inventors: Michael KERNS, Strongsville, OH (US); Alan GRESZLER, Strongsville, OH (US); Anton ZONNEVELD, Strongsville, OH (US); Shawn Lamont BRAXTON, South Euclid, OH (US)

(73) Assignee: LUMITEX, INC., Strongsville, OH (US)

(21) Appl. No.: 18/856,646

(22) PCT Filed: Jun. 22, 2023
(86) PCT No.: PCT/US2023/068877

§ 371 Date: Oct. 14, 2024

Related U.S. Application Data
(60) Provisional application No. 63/367,253, filed on Jun. 29, 2022.

(51) Int. Cl. A61N 5/06; A61B 5/00; A61B 5/01; A61B 5/08; A61B 5/145

(52) U.S. Cl. CPC A61N 5/0613; A61B 5/01; A61B 5/0816; A61B 5/14542; A61B 5/14546; A61B 5/4836; A61B 5/7264; A61B 5/7275; A61B 5/746; A61B 2560/0462; A61N 2005/0627; A61N 2005/063; A61N 2005/0642; A61N 2005/0645; A61N 2005/0652

(57) ABSTRACT

The present disclosure provides a controller for modulating phototherapeutic treatment of inflammatory conditions by modulating electromagnetic radiation output from a phototherapy apparatus based on respiratory data from a respiration sensor. The controller modulates a property of the electromagnetic radiation emitted by the light source based on the received respiratory data, such that at least one of: the phototherapy is decreased in response to a positive trend in the respiratory data or the phototherapy is increased in response to a negative trend in the respiratory data.

This International Application claims priority to U.S. Provisional Application No. 63/367,253 filed Jun. 29, 2022, which is hereby incorporated herein by reference in its entirety.

### TECHNICAL FIELD

The present disclosure relates generally to light emitting apparatus and more particularly to a light emitting apparatus for treating inflammatory conditions such as acute respiratory distress syndrome (ARDS).

### BACKGROUND

Acute respiratory distress syndrome (ARDS) leads to acute respiratory failure and is caused by a variety of factors. ARDS generally presents with progressive hypoxemia, dyspnea, and increased difficulty breathing. Patients often require mechanical ventilation and supplemental oxygen. ARDS is associated with significant morbidity and mortality and therapeutic strategies to mitigate the foregoing have resulted in limited translational success.

ARDS is induced by many factors, including bacterial and viral pneumonia, sepsis, inhalation of harmful substances, head, chest or other major injury, burns, blood transfusions, near drowning, aspiration of gastric contents, pancreatitis, intravenous drug use, and abdominal trauma.

ARDS is often associated with inflammation and fluid accumulation in the lungs, resulting in impairment of the lungs and less oxygen uptake into the bloodstream. This deprives organs of the oxygen required for normal function and viability. Severe shortness of breath, the main symptom of ARDS, usually develops within a few hours to a few days after the precipitating injury or infection.

### SUMMARY

One of the factors leading to the morbidity and mortality and ARDS is caused by a cytokine storm (also referred to as cytokine release syndrome). A cytokine storm is a life-threatening systemic inflammatory syndrome involving elevated levels of circulating cytokines and immune cell hyperactivation that can be triggered by various therapies, pathogens, cancers, autoimmune conditions, and monogenic disorders.

Phototherapy has been shown to alleviate the symptoms of ARDS and to increase blood oxygen levels. One proposed mechanism for this positive effect is that the majority of reactive oxygen species (ROS) (also referred to as free radicals) may be generated in the mitochondria under conditions of cellular stress. Phototherapy is thought to stimulate mitochondrial enzymes (e.g., cytochromes) to reduce the generation of ROS.

The present disclosure provides a controller of a phototherapy apparatus for modulating phototherapeutic treatment of inflammatory conditions by modulating electromagnetic radiation (also referred to as light) output based on physiological measurements including at least one of breathing rate or blood oxygen level

While a number of features are described herein with respect to embodiments of the invention; features described with respect to a given embodiment also may be employed in connection with other embodiments. The following description and the annexed drawings set forth certain illustrative embodiments of the invention. These embodiments are indicative, however, of but a few of the various ways in which the principles of the invention may be employed. Other objects, advantages, and novel features according to aspects of the invention will become apparent from the following detailed description when considered in conjunction with the drawings.

### BRIEF DESCRIPTION OF THE DRAWINGS

The annexed drawings, which are not necessarily to scale, show various aspects of the invention in which similar reference numerals are used to indicate the same or similar parts in the various views.

FIG. 1 is a block diagram of an exemplary embodiment of a phototherapy apparatus.

FIG. 2 is an exemplary plot of patient health depicting transition from a healthy baseline into acute respiratory distress syndrome (ARDS).

FIG. 3 is an exemplary plot of patient health from FIG. 2 when the patient receives phototherapy.

FIG. 4 is first exemplary plot of a positive trend in respiratory data and a corresponding decrease in the optical dose applied to a patient.

FIG. 5 is second exemplary plot of a positive trend in respiratory data and a corresponding decrease in the optical dose applied to the patient.

FIG. 6 is first exemplary plot of a negative trend in respiratory data and a corresponding increase in the optical dose applied to the patient.

FIG. 7 is second exemplary plot of a negative trend in respiratory data and a corresponding increase in the optical dose applied to the patient.

FIG. 8 shows an enhanced increase in the optical dose in FIG. 7 due to received blood test results.

FIG. 9 is a block diagram of an exemplary embodiment of the phototherapy apparatus including a temperature sensor.

FIG. 10 is a block diagram of a light source including light emitters and a pad.

FIG. 11 is a block diagram of an exemplary embodiment of a phototherapy apparatus including a covering.

FIG. 12 is an exemplary embodiment of a method performed using a controller for modulating patient illumination to treat an inflammatory condition using a phototherapy apparatus.

The present invention is now described in detail with reference to the drawings. In the drawings, each element with a reference number is similar to other elements with the same reference number independent of any letter designation following the reference number. In the text, a reference number with a specific letter designation following the reference number refers to the specific element with the number and letter designation and a reference number without a specific letter designation refers to all elements with the same reference number independent of any letter designation following the reference number in the drawings.

### DETAILED DESCRIPTION

The present disclosure provides a controller for modulating phototherapeutic treatment of inflammatory conditions by modulating electromagnetic radiation output from a phototherapy apparatus based on respiratory data from a respiration sensor. The controller modulates a property of the electromagnetic radiation emitted by the light source based on the received respiratory data, such that at least one of: the phototherapy is decreased in response to a positive trend in the respiratory data or the phototherapy is increased in response to a negative trend in the respiratory data.

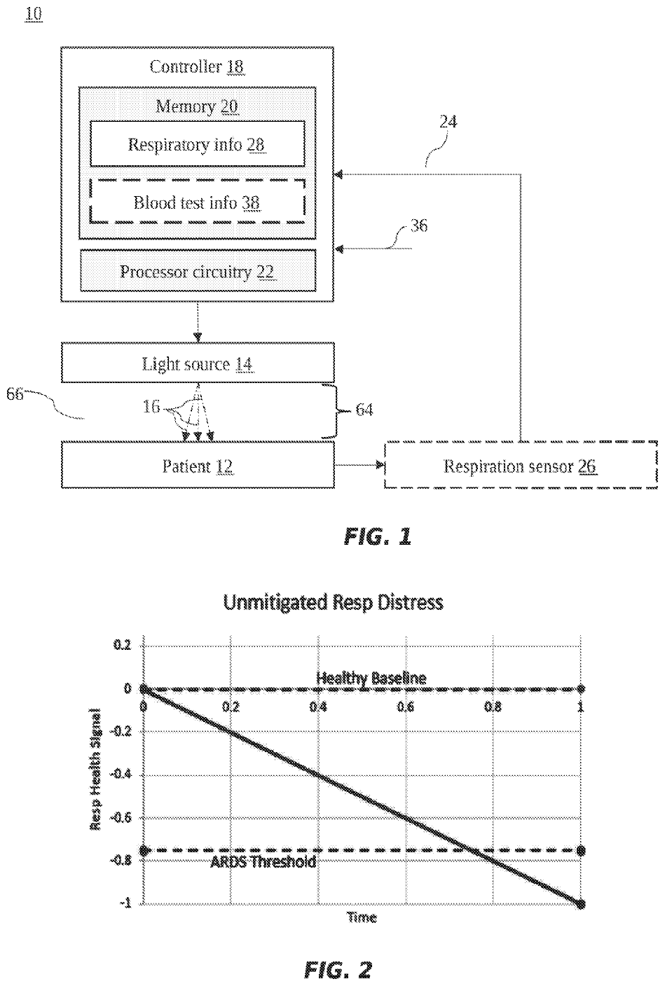

In the embodiments shown in FIG. 1, a phototherapy apparatus 10 for illuminating a patient 12 to treat an inflammatory condition is shown. The phototherapy apparatus 10 includes a light source 14 for emitting electromagnetic radiation 16 (also referred to as light). The phototherapy apparatus 10 also includes a controller 18 (also referred to as electronic controller) including memory 20 and processor circuitry 22. The processor circuitry 22 receives respiratory data 24 from a respiration sensor 26. The respiration sensor 26 senses and outputs respiratory data 24 based on at least one of a breathing rate or a blood oxygen level of the patient 12. Respiratory information 28 is stored in the memory 20 based on the received respiratory data 24. The processor circuitry 22 controls emission of the electromagnetic radiation 16 by the light source 14 based on the respiratory information 28 by modulating a property 30 of the electromagnetic radiation 16 emitted by the light source 14. The property 30 of the electromagnetic radiation 16 is modulated, such that (1) the property 30 is decreased in response to a positive trend in the respiratory information 28 over a duration of time and/or (2) the property 30 of the electromagnetic radiation being emitted is increased in response to a negative trend in the respiratory information 28 over the duration of time.

The phototherapy apparatus 10 may be used to treat any suitable inflammatory condition, such as acute respiratory distress syndrome (ARDS). For example, FIG. 2 shows patient transition from a healthy baseline into ARDS. In the exemplary figure, the patient starts at a healthy baseline and at time zero receives some insult (e.g., injury, disease, etc.). After time zero, health steadily declines until the patient passes an ARDS threshold. The trace called “Respiratory Health Signal” represents a merit function used to represent the patient's immediate health state. For example, the merit function may be a weighted mix of respiratory parameters, blood test results, heart rate, etc.

In FIG. 3, an exemplary graph of patient response to phototherapy is shown. FIG. 3 shows the same health trace and ARDS threshold as in FIG. 2. FIG. 3 also shows application of phototherapy (referred to in FIG. 3 as the device duty cycle). As in FIG. 2, the patient starts declining at time zero and phototherapy is applied with one property (e.g., full duty) as represented by a height of the “duty cycle” plot. In response to the phototherapy, the patient temporarily improves and stabilizes throughout application of the phototherapy (i.e., when the phototherapy apparatus 10 is in its ON cycle). When the phototherapy apparatus 10 stops delivering phototherapy (e.g., turns OFF to allow a rest period), ATP response returns to baseline and the patient's acute improvements fade until phototherapy is again applied by the phototherapy apparatus 10. In FIG. 3, there are two health states: one is the periodic change which represents acute response to our treatment; second is a slower more general improvement of patient health that comes with extended treatment. In FIG. 3, the periodic up/down gets closer and closer to baseline until the patient is recovered. Also, in FIG. 3, the property (e.g., duty cycle strength) decreases as patient health improves.

The respiration sensor 26 may be any suitable device for measuring a property of the patient's respiration. For example, the respiration sensor 26 may be a pulse oximeter, breathing rate sensor, spirometer, etc. The respiratory information 28 stored in the memory 20 may be a copy of the respiratory data 24 received from the respiration sensor 26. The respiratory information 28 may also be the result of processing performed on the respiratory data 24. For example, the respiratory information 28 may be the result of subsampling, compression, statistical processing of the respiratory data 24, etc.

The positive trend in the respiratory information 28 over the duration of time is at least one of (1) a decrease in a breathing rate of the patient over the duration of time greater than a breathing rate change threshold or (2) an increase in the blood oxygen level of the patient over the duration of time greater than a blood oxygen change threshold. Similarly, the negative trend in the respiratory information 28 over the duration of time is at least one of (1) a decrease in the breathing rate over the duration of time less than the breathing rate change threshold or (2) an increase in the blood oxygen level over the duration of time less than the bloody oxygen change threshold.

The blood oxygen change threshold and breathing rate change threshold may be any suitable rate of change indicating patient improvement. For example, the blood oxygen change threshold may be a predetermined rate of change identified as indicating a positive change in blood oxygen level (e.g., indicating that phototherapy previously applied by the phototherapy apparatus was effective). A change in the bloody oxygen level of less than the bloody oxygen change threshold indicates a negative change in bloody oxygen level (e.g., indicating that the phototherapy previously applied by the phototherapy apparatus was not effective).

Similarly, the breathing rate change threshold may be a predetermined rate of change identified as indicating a positive change in the breathing rate (e.g., indicating that phototherapy previously applied by the phototherapy apparatus was effective). A change in the breathing rate of less than the breathing rate change threshold indicates a negative change in breathing rate (e.g., indicating that the phototherapy previously applied by the phototherapy apparatus was not effective).

As described above, the processor circuitry 22 alters the property 30 of the electromagnetic radiation 16 being emitted based on the trend in the respiratory data 24 over a duration of time 32. The altered property 30 of the light 16 is at least one of optical dose, optical intensity, time duration of emission (e.g., an amount of time that light is emitted by the light source 14), fluence (i.e., time-integrated flux of radiation), wavelength, or optical power.

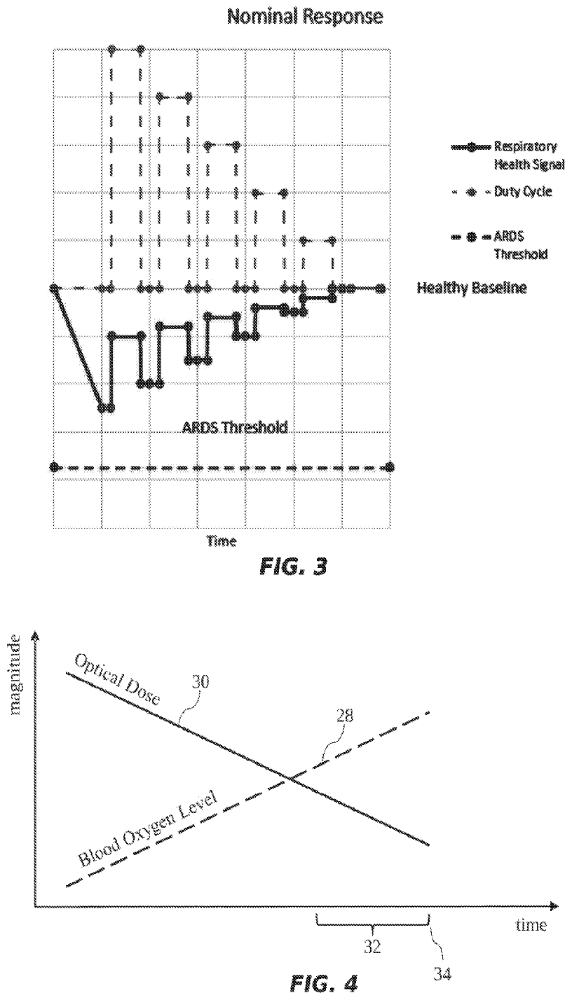

As shown in FIG. 4, the altered property 30 may be optical dose and the respiratory information 28 may be based on past and current blood oxygen levels. As shown, the altered property 30 may be inversely proportional to the respiratory information 28. That is, a positive change in the respiratory information 28 may result in a negative change in the property 30.

In one embodiment, the size of the change (i.e., the amount of the change) in the property 30 may be determined by the processor circuitry 22. That is, if a positive trend is found in the respiratory information 28, the processor circuitry 22 may cause a decrease in the property 30 of the light 16. The size of this decrease (i.e., the amount of the decrease in the property 30 of the light 16 being emitted) may be based on an amount of the positive change in the respiratory information 28 over the duration of time 32.

For example, the size of the positive trend over the duration of time 32 shown in FIG. 4 is smaller than the size of the positive trend over the same size duration of time 32 in FIG. 5. That is, the blood oxygen level in FIG. 4 improved more significantly (i.e., is higher at time point 34 at the end of the time duration 32) than the blood oxygen level in FIG. 5 at the same time point 34. Consequently, the optical dose in FIG. 4 may be decreased more significantly at the time point 34 than the optical dose in FIG. 5.

In this way, a larger amount of positive change in the property 30 over the duration of time 32 may result in a larger decrease in the property 30 of the light 16. Similarly, a smaller amount of positive change over the duration of time 32 may result in a smaller decrease in the property 30.

The duration of time 32 may be any suitable amount of time. For example, the time duration 32 may be an entire time the respiratory information 28 has been collected, six hours, 24 hours, one week, 30 days, etc.

Similar to the above-described decrease of the property 30 due to a positive trend, if a negative trend is found in the respiratory information 28, the processor circuitry 22 may cause an increase in the property 30 of the light 16. The size of this increase (i.e., the amount of the increase in the property 30 of the light 16 being emitted) may be based on an amount of the negative change in the respiratory information 28 over the duration of time 32.

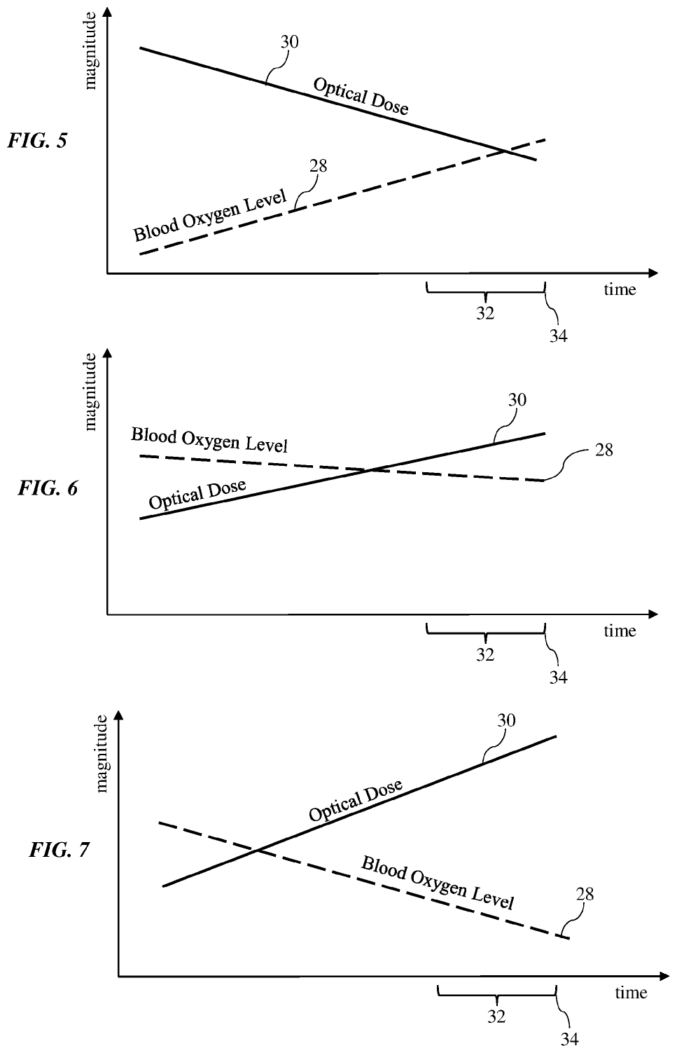

For example, the size of the negative trend over the duration of time 32 shown in FIG. 6 is smaller than the size of the negative trend over the same duration of time 32 in FIG. 7. That is, the blood oxygen level in FIG. 6 decreased less significantly than the blood oxygen level in FIG. 7 at the same time point 34. Consequently, the optical dose in FIG. 7 may be increased more significantly at the time point 34 than the optical dose in FIG. 6.

In this way, a larger amount of negative change in the property 30 over the duration of time 32 may result in a larger increase in the property 30 of the light 16. Similarly, a smaller amount of negative change over the duration of time 32 may result in a smaller increase in the property 30.

In one embodiment, the property 30 may be increased if there has not been a change (e.g., not statistically significant change) in the respiratory information 28. That is, a lack of change may be treated as a negative trend.

The feedback control of the property 30 of the electromagnetic radiation 16 based on the trend of the in the respiratory information 28 may use any suitable feedback scheme. For example, the feedback control may be a single-biomarker proportional feedback scheme (i.e., use one type of respiratory information 28 such as breathing rate). Alternatively, the feedback control may also use a weighted merit function that takes as an input multiple biomarkers simultaneously (e.g., breathing rate and oxygen levels).

The processor circuitry 22 may also receive blood test results 36 of the patient 12 including a measure of a compound in the blood. The measured compound may include at least one of Interleukin 6, Interleukin 9, or C-reactive protein (CRP). Blood test information 38 may be stored in the memory 18 based on the received blood test results 36. Based on the blood test information 38, the processor circuitry 22 may adjust at least one of: the amount of the increase of the property 30 of the emitted light 16 or the amount of the decrease of the property 30 of the emitted light 16.

Similar to the above description of the respiratory information 28, the blood test information 38 stored in the memory 20 may be a copy of the blood test results 36 received or a result of processing performed on the blood test results 36. For example, the blood test information 38 may be the result of subsampling, compression, statistical processing of the blood test results 36, etc.

The amount of the increase of the property 30 may be adjusted such that, when the blood test information 38 includes a positive indication due to a decrease in the measured compound over the period of time (e.g., indicating that the phototherapy applied using the phototherapy apparatus 10 has been effective), the processor circuitry 22 lessens the amount (i.e., the size) of the increase in the property 30. Alternatively or additionally, the amount of the increase of the property 30 may be adjusted such that, when the blood test information 38 includes a negative indication due to an increase in the measured compound over the period of time 32 (e.g., indicating the phototherapy has not been effective), the processor circuitry 22 escalates the amount of the increase in the property 30.

The amount of the decrease of the property 30 may be adjusted such that, when the blood test information 38 includes a positive indication due to a decrease in the measured compound over the period of time 34, the processor circuitry 22 escalates the amount of the decrease in the property 30. The amount of the decrease of the property 30 may be adjusted such that, when the blood test information 38 includes a negative indication due to an increase in the measured compound over the period of time 32, the amount of the decrease in the property 30 is lessened.

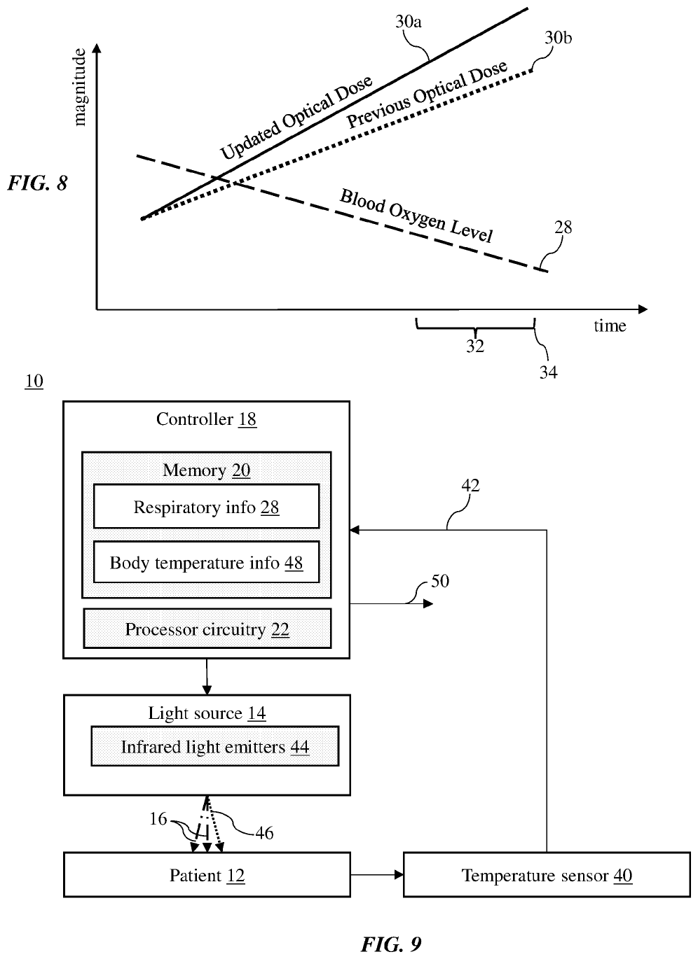

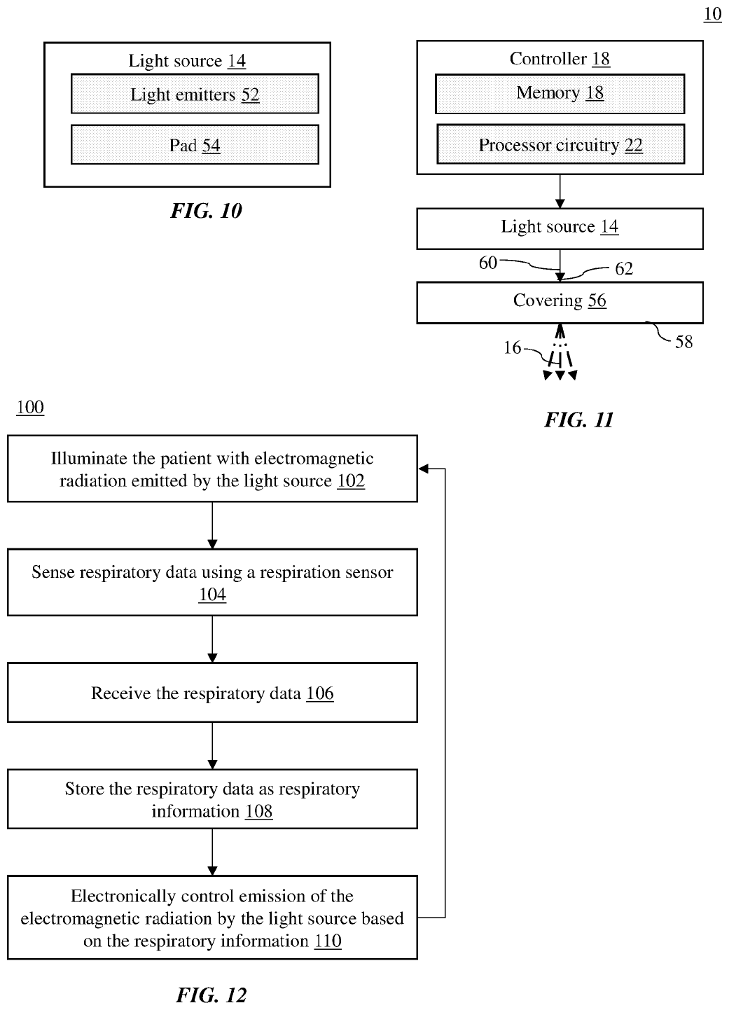

As an example, FIG. 7 may exemplify the amount of the decrease of the property 30 based on initial blood test information 38. Upon receiving updated blood test information 38 showing a negative trend in the measured compound (e.g., an increase in Interleukin 6), the processor circuitry 22 may modify the plot shown in FIG. 8 as shown. That is, the processor circuitry 22 may increase the change in optical dose by a larger amount (i.e., compared to FIG. 7) for a same change in respiratory information 28. In FIG. 8, the plot of optical dose 30b from FIG. 7 is repeated (dotted line) to show the change from the updated optical dose 30a (solid line) due to the negative trend in the blood test information 38.

The amount of change of the respiratory information 28 and/or the property 30 of the electromagnetic radiation 16 may be determined using any suitable method. For example, a fit (e.g., linear, quadratic, exponential etc.) may be applied to the respiratory information 28 and/or the property 30 and a term (also referred to as a coefficient) of the fit (e.g., slope) may be used as a measurement of the amount of change.

While FIGS. 4-8 represent the property 30 as a continuous line, the property 30 may be represented as a step function. That is, phototherapy may either be applied or may not be applied based on patient response (i.e., the trend in the respiratory information), such that the electromagnetic radiation 16 is delivered in discrete doses rather than a continuous line. For example, if a patient is not responding as expecting (i.e., based on the trend in the respiratory information) then phototherapy may be applied by causing the light source 14 to emit electromagnetic radiation 16. Conversely, if the patient is responding positively (i.e., based on the trend in the respiratory information) then phototherapy may not be applied by causing the light source 14 to stop emitting electromagnetic radiation 16.

Similarly, the respiratory information 28 is shown in FIGS. 4-8 as being linear. The respiratory information 28 may have a more stepwise path and then taper off after a maximal dose of electromagnetic radiation has been received.

Also, adding a biomarker of CRP could be helpful because it measures broad inflammation and could be used as a marker for systemic inflammation

Turning to the embodiment shown in FIG. 9, the phototherapy apparatus 10 may also include a temperature sensor 40 for measuring a body temperature of the patient 12. The temperature sensor 40 outputs body temperature data 42 based on the measured body temperature. The processor circuitry 22 may receive the body temperature data 42 from the temperature sensor 40 and store body temperature information 48 in the memory 18 based on the received body temperature data 42. The temperature sensor 40 may be any suitable device for measuring body temperature of the patient 12.

The body temperature information 48 stored in the memory 20 may be a copy of the body temperature data 42 received from the temperature sensor 40. The body temperature information 48 may also be the result of processing performed on the body temperature data 42. For example, the body temperature information 48 may be the result of subsampling, compression, statistical processing of the body temperature data 42, etc.

In this embodiment, the light source 14 may include infrared light emitters 44 for emitting infrared electromagnetic radiation 46. The processor circuitry 22 may control emission of the infrared electromagnetic radiation 46 by the infrared light emitters 44 based on the body temperature information 48. For example, emission of infrared electromagnetic radiation 46 by the infrared light emitters 44 may be increased for a negative trend of the body temperature information 48. Alternatively or additionally, emission of infrared electromagnetic radiation 46 by the infrared light emitters 44 may be decreased for a positive trend of the body temperature information 48.

A positive trend of the body temperature information 48 may refer to an increase in the measured body temperature of the patient 42 from a previous time point to a current time point (i.e., body temperature has increased over a duration of time). Similarly, a negative trend of the body temperature information 48 may refer to a decrease in the measured body temperature of the patient 42 from a previous time point to a current time point (i.e., body temperature has decreased over a duration of time).

The processor circuitry 22 may control emission of the infrared light emitters 44 based on a difference between the measured body temperature and a preset body temperature. For example, the preset body temperature may be 98.6° F. and the processor circuitry 22 may control the emission of infrared light 46 in proportion to the difference between the preset body temperature and the current body temperature. Because infrared light may also activate mitochondria, using red and infrared light may both activate mitochondria and manage thermal effects. In one example the optical dose output by the infrared light emitter 44 may decrease the closer the measured body temperature is to the preset body temperature.

The processor circuitry 22 may also monitor the respiratory information 28 for deviations outside of normal bounds. When a deviation outside of normal bounds is detected, the processor circuitry 22 may issue a notification 50 (e.g., indicating a potential cytokine storm). The normal bounds may include at least two of: a minimum oxygen level, a maximum oxygen level, a minimum breathing rate, or a maximum breathing rate. The normal bounds may also include patient body temperature above a given threshold (e.g., indicating a fever). The normal bounds may be received by the processor circuitry or may be determined by the processor circuitry based on the respiratory information 28. For example, a medical professional may supply the normal bounds to the processor circuitry 22. Alternatively, the processor circuitry 22 may determine values for the normal bounds based on statistical analysis of the respiratory information 28.

As shown in FIG. 11, the light source 14 may include light emitters 52 and a pad 54. The pad 54 may mechanically support the light emitters 52 such that, when the pad is laid on a skin surface of the patient 12, the light emitters 52 are oriented and positioned to illuminate the skin surface of the patient 12.

The pad 54 may be made of a biocompatible material having a hardness of 50 shore A or less. A softness of the pad 54 may depend on properties of the skin surface 32 and/or the target tissue 19. For example, the pad 54 softness may be sufficient for the pad 54 to conform to a contour of the skin surface of the patient 12.

The pad 54 may be any suitable material. In one example, the pad 54 may be made from silicone, urethane, polyethylene, or any material having a hardness of 50 shore A or softer. The pad 54 may be flexibly molded, rigid, or machined to match the contour of the skin surface. A portion of the pad 54 including the light emitting surface may have a hardness of 50 shore A or less to mitigate tissue damage that may be caused by more rigid structures.

The electromagnetic radiation 16 may be received via any skin surface of the patient 12. For example, the torso may be illuminated by the electromagnetic radiation 16. In another example, a burn patient may have non-damaged or less-damaged areas of skin illuminated (e.g., the legs or arms).

An interface material (such as an index matching gel) may be applied to the skin surface as an interface to the body. The interface material may be thermal conductive and mediate skin temperature. The interface material may be index matching to enhance optical coupling. The interface material may be used to mechanically cushion the tissue and/or to adhere to the surface tissue.

Turning to FIG. 11, the phototherapy apparatus 10 may further include a covering 56 optically connected to the light source 14 and configured to receive the electromagnetic radiation 16 emitted from the light source 14. The covering 56 may include a light emitting surface 58 and the covering may emit the received electromagnetic radiation 16 from the light emitting surface 58. The covering 56 may be at least one of a blanket or a garment (e.g., a vest or shirt).

The light source 14 may be optically connected to the covering via a light guide 60. The light guide 60 may interface with the covering 56 via a ferrule 62. To avoid bedsores and to improve patient comfort, the ferrule 62 may have a hardness of 50 shore A or less and/or may be located at a distance of at least six inches from the covering 56. For example, the ferrule 62 may be made from silicone cast over optical fibers extending from the covering 56. In one embodiment, the light source 14 may be a linear array of light emitting diodes (LEDs) and the ferrule 62 may have a linear shape complimentary to the linear array of LEDs.

As shown in FIG. 3, the light source 14 may direct the emitted electromagnetic radiation 16 onto the patient 12 from a distance 64, such that the emitted electromagnetic radiation 16 passes through an airgap 66 between the patient 12 and the light source 14.

The light source 14 may be any source of electromagnetic radiation 16 (also referred to as light). For example, the light source 14 may be an external light box mechanically separated from the covering 56 or pad 54 and optically coupled to the covering 56 or pad 54 via the light guide 60. In another example, the light source 14 may be a projecting light source (such as a goose neck lamp) that directs light through the air to illuminate the patient 12. In still another example, the light source includes multiple light emitters physically supported adjacent to a skin surface of the patient 12.

As described above, the light source 14 may include light emitters 52. The light emitters 52 may be any suitable structure for emitting electromagnetic radiation 16. For example, the light emitters 52 may include one or more light emitting diodes (LEDs), organic LEDs (OLEDs), microLEDs, laser diodes, mini-LED, quantum dot (QD)-conversion, phosphor conversion, excimer lamps, multi-photon combination, or SLM wavefront manipulation.

The electromagnetic radiation 16 may have any suitable properties for treating inflammatory conditions. For example, the electromagnetic radiation 16 may include any wavelength of electromagnetic radiation 16, such as infrared (e.g., 800-1000 nm), red (e.g., 620-720), and blue (e.g., 405 nm, 450 nm). The light source may emit different wavelengths of light sequentially or simultaneously. In one embodiment, the electromagnetic radiation 16 includes blue light for reducing scarring, and 405 nm light or ultraviolet (UV) light to reduce infection. In one embodiment, the electromagnetic radiation 16 includes mixed wavelength to effectively activate mitochondria while controlling temperature, reducing viral load, and minimizing scarring.

Turning to FIG. 12, an embodiment of a method 100 performed using a controller 18 including processor circuitry 22 is shown for modulating patient illumination to treat an inflammatory condition using a phototherapy apparatus 10. In step 102, the patient 12 is illuminated with electromagnetic radiation 16 emitted by the light source 14 of the phototherapy apparatus 10. In step 104, respiratory data 24 is sensed using a respiration sensor 26. In step 106, respiratory data 24 is received by the processor circuitry 22. In step 108, respiratory information 28 is stored in the memory 20 of the controller 18. In step 110, the processor circuitry 22 controls emission of the electromagnetic radiation 16 by the light source 14 based on the respiratory information 28 by modulating a property 30 of the electromagnetic radiation 16 emitted by the light source 14. As described above, the property of the electromagnetic radiation being emitted is decreased in response to a positive trend in the respiratory information over a duration of time. Similarly, the property of the electromagnetic radiation being emitted is increased in response to a negative trend in the respiratory information over the duration of time.

As shown in FIG. 12 with the method 100 returning to previous steps following step 110, the respiratory information 28 may be used as a feedback loop to control electromagnetic radiation 16 by the light source 14.

The processor circuitry 22 may have various implementations. For example, the processor circuitry 22 may include any suitable device, such as a processor (e.g., CPU), programmable circuit, integrated circuit, memory and I/O circuits, an application specific integrated circuit, microcontroller, complex programmable logic device, other programmable circuits, or the like. The processor circuitry 22 may also include a non-transitory computer readable medium, such as random-access memory (RAM), a read-only memory (ROM), an erasable programmable read-only memory (EPROM or Flash memory), or any other suitable medium. Instructions for performing the method described below may be stored in the non-transitory computer readable medium and executed by the processor circuitry 22. The processor circuitry 22 may be communicatively coupled to the computer readable medium and network interface through a system bus, mother board, or using any other suitable structure known in the art.

The memory 20 may be any suitable computer readable medium. For example, the memory 20 may be one or more of a buffer, a flash memory, a hard drive, a removable media, a volatile memory, a non-volatile memory, a random access memory (RAM), or other suitable device. In a typical arrangement, the computer readable medium 20 may include a non-volatile memory for long term data storage and a volatile memory that functions as system memory for the processor 20. The computer readable medium 20 may exchange data with the circuitry over a data bus. Accompanying control lines and an address bus between the computer readable medium 20 and the circuitry also may be present. The computer readable medium 20 is considered a non-transitory computer readable medium.

All ranges and ratio limits disclosed in the specification and claims may be combined in any manner. Unless specifically stated otherwise, references to “a,” “an,” and/or “the” may include one or more than one, and that reference to an item in the singular may also include the item in the plural.

Although the invention has been shown and described with respect to a certain embodiment or embodiments, equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A phototherapy apparatus for illuminating a patient to treat an inflammatory condition, the phototherapy apparatus comprising:
a light source configured to emit electromagnetic radiation;
a controller including memory and processor circuitry configured to:
receive respiratory data from a respiration sensor configured to sense and output respiratory data based on at least one of a breathing rate or a blood oxygen level of the patient;
store respiratory information in the memory based on the received respiratory data;
receive blood test results of the patient comprising a measure of a compound in the blood, wherein the measured compound includes at least one of Interleukin 6, Interleukin 9, or C-reactive protein (CRP);
store blood test information in the memory based on the received blood test results; and
control emission of the electromagnetic radiation by the light source based on the respiratory information and the blood test information by modulating a property of the electromagnetic radiation emitted by the light source, such that:
the property of the electromagnetic radiation being emitted is decreased in response to a positive trend in the respiratory information over a duration of time, wherein:
the positive trend in the respiratory information over the duration of time comprises at least one of:
a decrease in a breathing rate of the patient over the duration of time greater than a breathing rate change threshold; or
an increase in the blood oxygen level of the patient over the duration of time greater than a blood oxygen change threshold; and
when the blood test information includes a positive indication due to a decrease in the measured compound over the period of time, escalating the amount of the decrease in the property of the electromagnetic radiation being emitted in response to the positive trend in the respiratory information over the duration of time; or
when the blood test information includes a negative indication due to an increase in the measured compound over the period of time, lessening the amount of the decrease in the property of the electromagnetic radiation being emitted in response to the positive trend in the respiratory information over the duration of time;
the property of the electromagnetic radiation being emitted is increased in response to a negative trend in the respiratory information over the duration of time, wherein:
the negative trend in the respiratory information over the duration of time comprises at least one of:
a decrease in the breathing rate over the duration of time less than the breathing rate change threshold; or
an increase in the blood oxygen level over the duration of time less than the bloody oxygen change threshold; and
when the blood test information includes a positive indication due to a decrease in the measured compound over the period of time, lessening the amount of the increase in the property of the electromagnetic radiation being emitted in response to the negative trend in the respiratory information over the duration of time; or
when the blood test information includes a negative indication due to an increase in the measured compound over the period of time, escalating the amount of the increase in the property of the electromagnetic radiation being emitted in response to the negative trend in the respiratory information over the duration of time;
wherein the property of the electromagnetic radiation comprises at least one of optical dose, optical intensity, time duration of emission, fluence, wavelength, or optical power.

2. The phototherapy apparatus of claim 1, wherein:
an amount of the decrease in the property of the electromagnetic radiation being emitted in response to the positive trend in the respiratory information over the duration of time is determined by the processor circuitry based on an amount of the positive change in the respiratory information over the duration of time, such that:
a larger amount of the positive change over the duration of time results in a larger decrease in the property of the electromagnetic radiation being emitted; and
a smaller amount of the positive change over the duration of time results in a smaller decrease in the property of the electromagnetic radiation being emitted; and
an amount of the increase in the property of the electromagnetic radiation being emitted in response to the negative trend in the respiratory information over the duration of time is determined by the processor circuitry based on an amount of the negative change in the respiratory information over the duration of time, such that:
a larger amount of the negative change over the duration of time results in a larger increase in the property of the electromagnetic radiation being emitted; and
a smaller amount of the negative change over the duration of time results in a smaller increase in the property of the electromagnetic radiation being emitted.

3. (canceled)

4. The phototherapy apparatus of claim 1, further comprising a temperature sensor configured to measure a body temperature of the patient and output body temperature data based on the measured body temperature, wherein:
the light source includes infrared light emitters configured to emit infrared electromagnetic radiation;
the processor circuitry is configured to:
receive the body temperature data from the temperature sensor;
store body temperature information in the memory based on the received body temperature data;
control emission of the infrared electromagnetic radiation by the infrared light emitters, such that at least one:
emission of infrared electromagnetic radiation by the infrared light emitters is increased for a negative trend of the body temperature information; or
emission of infrared electromagnetic radiation by the infrared light emitters is decreased for a positive trend of the body temperature information.

5. The phototherapy apparatus of claim 1, wherein:
the processor circuitry is further configured to:
monitor the respiratory information for deviations outside of normal bounds; and
when a deviation outside of normal bounds is detected, issue a notification of a potential cytokine storm;
the normal bounds comprise at least two of a minimum oxygen level, a maximum oxygen level, a minimum breathing rate, or a maximum breathing rate; and
the normal bounds are received by the processor circuitry or are determined by the processor circuitry based on the respiratory information.

6. The phototherapy apparatus of claim 1, wherein:
the light source includes light emitters and a pad;
the pad is configured to mechanically support the light emitters, such that when the pad is laid on a skin surface of the patient, the light emitters are oriented and positioned to illuminate the skin surface of the patient.

7. The phototherapy apparatus of claim 6, wherein the pad is made of a biocompatible material having a hardness of 50 shore A or less.

8. The phototherapy apparatus of claim 1, further comprising a covering optically connected to the light source and configured to receive the electromagnetic radiation emitted from the light source;
wherein the covering includes a light emitting surface and the covering is configured to emit the received electromagnetic radiation from the light emitting surface.

9. The phototherapy apparatus of claim 8, wherein:
the light source is optically connected to the covering via a light guide;
the light guide interfaces with the covering via a ferrule; and
to avoid bedsores, the ferrule at least one of:
has a hardness of 50 shore A or less; or
is located at a distance of at least six inches from the covering.

10. The phototherapy apparatus of claim 8, wherein the covering comprises at least one of a blanket or a garment.

11. The phototherapy apparatus of claim 1, wherein the light source directs the emitted electromagnetic radiation onto the patient from a distance, such that the emitted electromagnetic radiation passes through an airgap between the patient and the light source.

12. The phototherapy apparatus of claim 1, wherein the inflammatory condition is acute respiratory distress syndrome (ARDS).

13. A controller of a phototherapy apparatus including a light source for illuminating a patient to treat an inflammatory condition, the controller including:
memory and processor circuitry configured to:
receive respiratory data from a respiration sensor of the phototherapy apparatus, wherein the respiration sensor comprises at least one of a breathing rate sensor or a blood oxygen sensor;
store respiratory information in the memory based on the received respiratory data;
receive blood test results of the patient comprising a measure of a compound in the blood, wherein the measured compound includes at least one of Interleukin 6, Interleukin 9, or C-reactive protein (CRP);
store blood test information in the memory based on the received blood test results; and
control emission of electromagnetic radiation by the light source based on the respiratory information and the blood test information by modulating a property of the electromagnetic radiation emitted by the light source, such that:
the property of the electromagnetic radiation being emitted is decreased in response to a positive trend in the respiratory information over a duration of time, wherein:
the positive trend in the respiratory information over the duration of time comprises at least one of
a decrease in a breathing rate of the patient over the duration of time greater than a breathing rate change threshold; or
an increase in the blood oxygen level of the patient over the duration of time greater than a blood oxygen change threshold; and
when the blood test information includes a positive indication due to a decrease in the measured compound over the period of time, escalating the amount of the decrease in the property of the electromagnetic radiation being emitted in response to the positive trend in the respiratory information over the duration of time; or
when the blood test information includes a negative indication due to an increase in the measured compound over the period of time, lessening the amount of the decrease in the property of the electromagnetic radiation being emitted in response to the positive trend in the respiratory information over the duration of time; and
the property of the electromagnetic radiation being emitted is increased in response to a negative trend in the respiratory information over the duration of time, wherein:
the negative trend in the respiratory information over the duration of time comprises at least one of:
a decrease in the breathing rate over the duration of time less than the breathing rate change threshold; or
an increase in the blood oxygen level over the duration of time less than the bloody oxygen change threshold;
when the blood test information includes a positive indication due to a decrease in the measured compound over the period of time, lessening the amount of the increase in the property of the electromagnetic radiation being emitted in response to the negative trend in the respiratory information over the duration of time; or
when the blood test information includes a negative indication due to an increase in the measured compound over the period of time, escalating the amount of the increase in the property of the electromagnetic radiation being emitted in response to the negative trend in the respiratory information over the duration of time;
wherein the property of the electromagnetic radiation comprises at least one of optical dose, optical intensity, time duration of emission, fluence, wavelength, or optical power.

14. The controller apparatus of claim 13, wherein:
an amount of the decrease in the property of the electromagnetic radiation being emitted in response to the positive trend in the respiratory information over the duration of time is determined based on an amount of the positive change in the respiratory information over the duration of time, such that:
a larger amount of the positive change over the duration of time results in a larger decrease in the property of the electromagnetic radiation being emitted; and
a smaller amount of the positive change over the duration of time results in a smaller decrease in the property of the electromagnetic radiation being emitted; and
an amount of the increase in the property of the electromagnetic radiation being emitted in response to the negative trend in the respiratory information over the duration of time is determined based on an amount of the negative change in the respiratory information over the duration of time, such that:
a larger amount of the negative change over the duration of time results in a larger increase in the property of the electromagnetic radiation being emitted; and
a smaller amount of the negative change over the duration of time results in a smaller increase in the property of the electromagnetic radiation being emitted; and

15. (canceled)

16. The controller of claim 13, further comprising a temperature sensor configured to measure a body temperature of the patient and output body temperature data based on the measured body temperature, wherein:
the light source includes infrared light emitters configured to emit infrared electromagnetic radiation;
the processor circuitry is configured to:
receive the body temperature data from the temperature sensor;
store body temperature information in the memory based on the received body temperature data;
control emission of the infrared electromagnetic radiation by the infrared light emitters, such that at least one:
emission of infrared electromagnetic radiation by the infrared light emitters is increased for a negative trend of the body temperature information; or
emission of infrared electromagnetic radiation by the infrared light emitters is decreased for a positive trend of the body temperature information.

17. The controller of claim 13, wherein:
the processor circuitry is further configured to:
monitor the respiratory information for deviations outside of normal bounds; and
when a deviation outside of normal bounds is detected, issue a notification of a potential cytokine storm;
the normal bounds comprise at least two of a minimum oxygen level, a maximum oxygen level, a minimum breathing rate, or a maximum breathing rate; and
the normal bounds are received by the processor circuitry or are determined by the processor circuitry based on the respiratory information.

18. A method performed using a controller for modulating patient illumination to treat an inflammatory condition using a phototherapy apparatus, the method including:
illuminating the patient with electromagnetic radiation emitted by a light source of the phototherapy apparatus;
sensing as respiratory data at least one of a breathing rate or a blood oxygen level of the patient using a respiration sensor;
receiving the respiratory data by processor circuitry of the controller;
storing respiratory information in memory of the controller based on the received respiratory data;
receiving with the processor circuitry blood test results of the patient comprising a measure of a compound in the blood, wherein the measured compound includes at least one of Interleukin 6, Interleukin 9, or C-reactive protein (CRP);
storing blood test information in the memory based on the received blood test results;
the processor circuitry controlling emission of the electromagnetic radiation by the light source based on the respiratory information and the blood test information by modulating a property of the electromagnetic radiation emitted by the light source, such that:
the property of the electromagnetic radiation being emitted is decreased in response to a positive trend in the respiratory information over a duration of time, wherein:
the positive trend in the respiratory information over the duration of time comprises at least one of:
a decrease in a breathing rate of the patient over the duration of time greater than a breathing rate change threshold; or
an increase in the blood oxygen level of the patient over the duration of time greater than a blood oxygen change threshold; and
when the blood test information includes a positive indication due to a decrease in the measured compound over the period of time, escalating the amount of the decrease in the property of the electromagnetic radiation being emitted in response to the positive trend in the respiratory information over the duration of time; or
when the blood test information includes a negative indication due to an increase in the measured compound over the period of time, lessening the amount of the decrease in the property of the electromagnetic radiation being emitted in response to the positive trend in the respiratory information over the duration of time; and
the property of the electromagnetic radiation being emitted is increased in response to a negative trend in the respiratory information over the duration of time, wherein:
the negative trend in the respiratory information over the duration of time comprises at least one of:
a decrease in the breathing rate over the duration of time less than the breathing rate change threshold; or
an increase in the blood oxygen level over the duration of time less than the bloody oxygen change threshold; and
when the blood test information includes a positive indication due to a decrease in the measured compound over the period of time, lessening the amount of the increase in the property of the electromagnetic radiation being emitted in response to the negative trend in the respiratory information over the duration of time; or
when the blood test information includes a negative indication due to an increase in the measured compound over the period of time, escalating the amount of the increase in the property of the electromagnetic radiation being emitted in response to the negative trend in the respiratory information over the duration of time
wherein the property of the electromagnetic radiation comprises at least one of optical dose, optical intensity, time duration of emission, fluence, wavelength, or optical power.

19. The method of claim 18, wherein:
an amount of the decrease in the property of the electromagnetic radiation being emitted in response to the positive trend in the respiratory information over the duration of time is determined by the processor circuitry based on an amount of the positive change in the respiratory information over the duration of time, such that:
a larger amount of the positive change over the duration of time results in a larger decrease in the property of the electromagnetic radiation being emitted; and
a smaller amount of the positive change over the duration of time results in a smaller decrease in the property of the electromagnetic radiation being emitted; and
an amount of the increase in the property of the electromagnetic radiation being emitted in response to the negative trend in the respiratory information over the duration of time is determined by the processor circuitry based on an amount of the negative change in the respiratory information over the duration of time, such that:
a larger amount of the negative change over the duration of time results in a larger increase in the property of the electromagnetic radiation being emitted; and
a smaller amount of the negative change over the duration of time results in a smaller increase in the property of the electromagnetic radiation being emitted; and

20. (canceled)

21. The method of claim 18, further comprising:
measuring a body temperature of the patient using a temperature sensor of the phototherapy apparatus and outputting body temperature data based on the measured body temperature;
the processor circuitry receiving the body temperature data from the temperature sensor;
storing body temperature information in the memory based on the received body temperature data; and
the processor circuitry controlling emission of infrared electromagnetic radiation by infrared light emitters of the light source, such that at least one:
emission of infrared electromagnetic radiation by the infrared light emitters is increased for a negative trend of the body temperature information; or
emission of infrared electromagnetic radiation by the infrared light emitters is decreased for a positive trend of the body temperature information.
